(19) United States
(12) Patent Application Publication
(10) Pub. No.: US 2025/0319309 A1
(43) Pub. Date: Oct. 16, 2025

(54) VAGUS NERVE STIMULATION APPLICATOR

(71) Applicant: Wesley G. Bradford, Rancho Palos Verdes, CA (US)

(72) Inventor: Wesley G. Bradford, Rancho Palos Verdes, CA (US)

(21) Appl. No.: 19/038,440

(22) Filed: Jan. 27, 2025

Related U.S. Application Data
(60) Provisional application No. 63/634,730, filed on Apr. 16, 2024.

(51) Int. Cl. A61N 1/36; A61N 1/04

(52) U.S. Cl. CPC A61N 1/36014; A61N 1/0456; A61N 1/0472

(57) ABSTRACT

A hand-held vagus nerve stimulator includes a housing having a proximal body and a distal cap. The distal cap has a distal surface configured to be placed on the carotid-pulse area of the user's neck. A pair of electrodes extend through the distal cap and a portion of the proximal body, and a pair of lead wires are captured between the proximal body and the distal cap. The size of the electrodes and the distance between them is optimized for use of the carotid-pulse area of the user's neck, and the lead wires are configured to be coupled to a programmable TENS unit.

### CROSS-REFERENCE TO RELATED APPLICATIONS

This application claims the benefit under 35 U.S.C. § 119 of U.S. provisional application No. 63/634,730, filed Apr. 16, 2024, entitled “VAGUS NERVE STIMULATOR,” the contents of which are incorporated herein by reference.

### BACKGROUND OF THE INVENTION

### 1. Field of the Invention

The present disclosure relates in general to nerve stimulation devices and, more particularly, to a non-invasive handheld vagal nerve stimulation applicator for connection to commercially available transcutaneous nerve stimulator (TENS) units.

### 2. Background Art

TENS units are used by chiropractors and physical therapists for passing a weak electrical current through the skin (transcutaneous), to stimulate nerves for therapeutic purposes. TENS devices stimulate local sensory nerves, and to some extent also peripheral motor nerves. The stimulation causes multiple mechanisms in the central nervous system to modulate the sense of pain. TENS effects are believed to result from stimulating competing sensory neurons at the pain perception gate in the nervous system, and from stimulating the nervous system's opiate pain-response. Common uses for TENS include musculoskeletal and neck/back pain, painful diabetic and other neuropathies, and menstrual and labor pain.

TENS units are usually connected to the skin using one or two pairs of conductive gel-pad electrodes. Most battery-powered TENS units can be adjusted to modulate pulse width, frequency, and intensity, according to the desired treatment modality. Generally, TENS frequencies over 50 Hz affect primarily sensory nerves, while frequencies below 10 Hz and with higher intensity produce primarily motor-nerve stimulation and muscle contraction. Many TENS units now use mixed frequency modes, to alleviate any tolerance to repeated use. The intensity of stimulation should be mild enough for comfort, consistent with producing the greatest pain relief.

Potential benefits of TENS treatment include safety, low cost, ability to self-administer, and availability without a medical prescription. TENS has been found to be safe compared to pharmaceutical medications for treating pain. Potential side effects include skin itching and mild redness near the conductive gel-pad electrodes. Precautions are recommended for pregnancy, epilepsy, active malignancy, vein thrombosis, damaged skin, or frailty. Contraindications include presence of implanted electronic medical devices such as pacemakers or cardio-defibrillators.

Vagal nerve stimulation (VNS) is a specialized form of transcutaneous electrical nerve stimulation applied to the vagus nerve (10 cranial nerve), which passes down the through neck with one branch on each side of the trachea next to the carotid artery and carries motor and sensory signals in both directions between the brain and the body organs. Because the vagus nerve is associated with many different functions and regions of the brain, clinical research has been done to determine the usefulness of VNS in a variety of illnesses, including anxiety disorders, obesity, alcohol addiction, chronic heart failure, prevention of cardiac arrhythmias, autoimmune diseases, irritable bowel syndrome, Alzheimer's disease, Parkinson's disease, hypertension, chronic pain, inflammatory disorders, fibromyalgia, migraines, depression, and obesity.

Implantable VNS devices have been approved by the US Food and Drug Administration (FDA) for treating epilepsy, depression, and stroke recovery. In addition, a handheld noninvasive VNS has been approved for treatment of migraine and episodic cluster headaches.

Unfortunately, existing commercially available TENS units are not suitable for use on the vagus nerve, since they are equipped with conducive gel-pad electrodes which are far too large (typically 1.25-2.0 inches in diameter) for placement on the neck. Furthermore, the sticky pads are messy and can be inconvenient to use. Alternatively, since a branch of the vagus nerve supplies part of the external ear, some TENS units provide ear clip electrodes which can be placed on the ear lobe, tragus, or cymba conchae, but these are difficult to place on the ear correctly, and have been shown to be somewhat less effective than VNS on the neck.

Several self-contained VNS devices are commercially available, but these suffer from several shortcomings. For instance, some of the devices are only available by prescription and/or subscription, and are preprogrammed to deliver fixed sine wave pulses for a fixed amount of time, giving users no ability to change their treatment in view of their individual needs or if future research suggests that alternate settings are preferable. Even those devices which are programmable offer a limited amount of customization; for example, they may allow the user to change pulse intensity and duration, but not waveform. Furthermore, the self-contained VNS devices contain batteries and electronic circuitry which may increase the size and of the device's housing, making it awkward to grip by hand. Finally, and perhaps most importantly, these devices are considerably more expensive than general-purpose TENS units.

### SUMMARY

The present disclosure relates to a hand-held vagus nerve stimulator including a housing having a proximal body and a distal cap. The distal cap has a distal surface configured to be placed on the carotid-pulse area of a user's neck. A pair of electrodes extend through the distal cap and a portion of the proximal body, and a pair of lead wires are captured between the proximal body and the distal cap. The size of the electrodes and the distance between them is optimized for use on the carotid-pulse area of the user's neck, and the lead wires are configured to be coupled to a programmable TENS unit.

In one aspect of the disclosure, the distal surface of the distal cap is planar.

In another aspect of the disclosure, the housing has a predetermined height H₁ and the distal surface has a predetermined length L₁, where L₁ is greater than H₁.

In still another aspect of the disclosure, a pair of cavities and a pair of channels are formed on the distal surface of the proximal body. Each cavity is configured to receive and retain a proximal portion of the one the electrodes, and each channel communicates with one of the electrodes and is configured to receive and retain one of the lead wires.

In another aspect of the disclosure, the distal cap is sealed to the proximal body.

In yet another aspect of the disclosure the proximal body has a distal portion and a proximal portion. The distal portion has a predetermined width W₁, and the proximal portion is configured as a finger grip sized and shaped to be easily clasped between an index finger and a thumb. The finger grip has a predetermined width W₁ that is less than W₂. In one example, W₁ is approximately half of W₂

In another aspect of the disclosure, each of the lead wires has a distal end captured between the proximal portion of one of the electrodes and a wall of the surrounding cavity.

In still another aspect of the disclosure, each of the electrodes includes a solid cylindrical main portion and a pair of diametrically opposed, proximally extending ears, and the distal end of each lead wire is captured between one of the ears and a wall of the surrounding cavity

### BRIEF DESCRIPTION OF THE DRAWINGS

FIG. 1 is a perspective view of a vagal nerve stimulation (VNS) applicator according to the present disclosure.

FIG. 2 is a front view of FIG. 1.

FIG. 3 is a side view of FIG. 1

FIG. 4 is a bottom plan view of the proximal body of the VNS applicator.

FIG. 5 is a view of an electrode.

FIG. 6 is a front view of the distal cap of the applicator.

FIG. 7 is a top view of the distal cap of the applicator.

FIG. 8 is a perspective view showing the elements of the applicator in exploded relationship to one another.

FIG. 9 shows the applicator connected to an over-the counter (OTC) programmable TENS unit.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

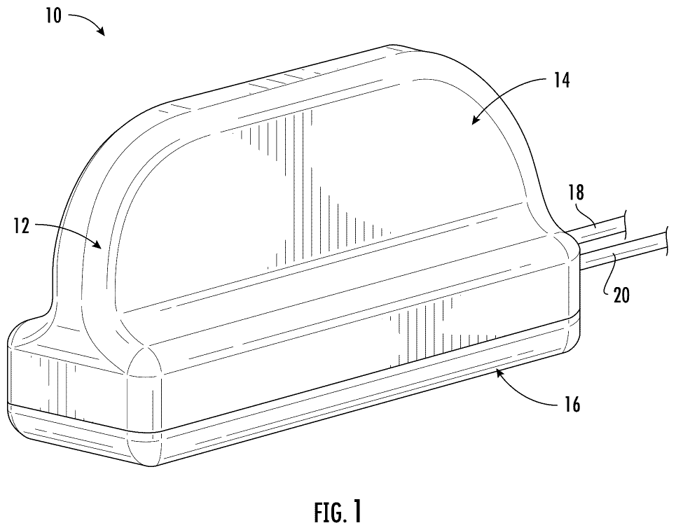

FIG. 1 is a perspective view showing a vagal nerve stimulation (VNS) applicator 10 designed to be placed on the anterior neck of a user. The VNS includes a housing 12 having an proximal body 14 and a distal cap 16. The proximal body 14 and distal cap are preferably injection-molded from a plastic material having anti-static properties. Since the applicator 10 will not need to withstand sterilizer treatment, the material need not be corrosion or temperature resistant.

A pair of standard-sized, insulated, stranded cables or lead wires 18, 20 extend through openings in a side wall of the proximal body 14 body to connect the VNS applicator 10 to a commercial programmable non-prescription TENS unit.

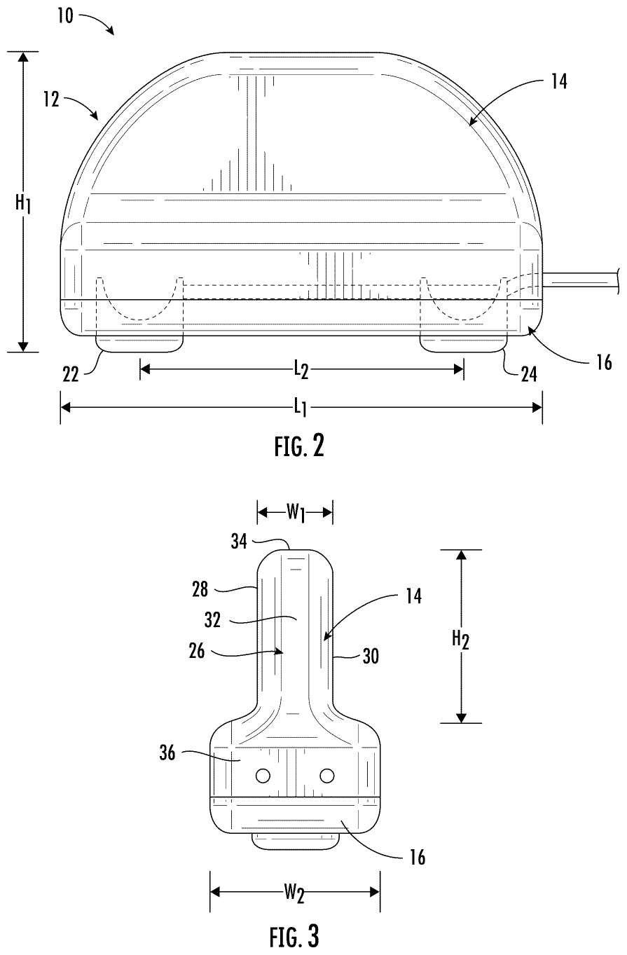

As seen in FIG. 2, the housing 12 has a length L₁ and a height H₁, where the length L₁ is greater than the height H₁. In one example, H₁ may be slightly less than half of L₁. For instance, H₁ may be 0.872″ and L₁ may be 1.625″. A pair of cup-shaped electrodes 22, 24 extend through openings in the bottom cap 16 and are secured to the lead wires 18, 20. The electrodes 22, 24, which are preferably made from a metal such as chrome-plated copper, are separated from one another by a distance L₂ which is approximately equal to the distance between the two branches of the vagus nerve at the carotid pulse area. For instance, L₂ may be in the range of 1.0″ to 1.25″.

As seen in FIG. 3, a proximal portion of the proximal body 12 is formed as a finger grip 26 having planar front and rear walls 28, 30, curved side walls 32, and a narrow, planar top wall 34. The dimensions of the finger grip 26 are selected to make it convenient and comfortable to grasp between a thumb and a finger. For instance, it may have a width W₁ of about 0.25″ to 0.375″ and a height H₂ of about 0.5″ to 0.625″ (or about two-thirds of the total height H₁ of the housing 12). At its distal end, the finger grip 26 joins a broader base 36 having the same width W₂ as the bottom cap 16, where W₂ is about 0.5″ to 0.625.″

FIG. 4 shows the bottom surface 38 of the base 36, which includes annular cavities 40, 42 for receiving the upper portions of the cup-shaped electrodes and narrow channels 44, 46 for receiving the lead wires.

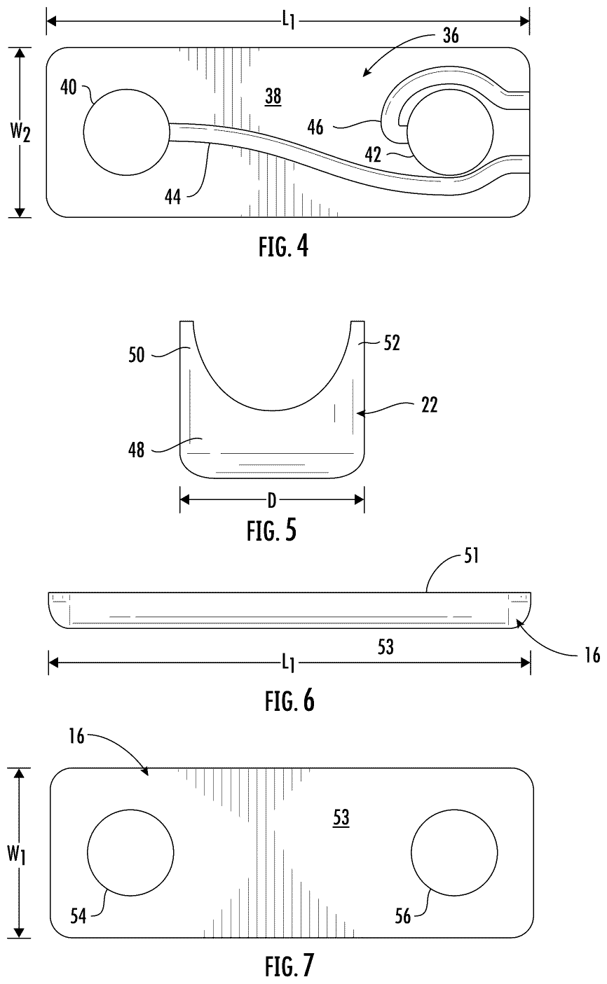

FIG. 5 is an enlarged view of a cup-shaped electrode 22. The electrode 22, which is stamped from a metal such as copper, includes a solid cylindrical main portion 48 and two diametrically opposed, proximally extending ears 50, 52. The outer diameter D of the main portion 48 is optimized for placement over the carotid-pulse area of a user's neck and may be approximately equal to the width W₁ of the finger grip 26; for instance, D may be in the range of about 0.25″ to about 0.375″, and most preferably is about 0.3125.″ The height h of the electrode 22 including the ears 50, 52 may be about equal to or slightly less than the outer diameter D of the main portion, as needed for secure connection to the wire strands in the proximal body.

FIGS. 6 and 7 are front and top views, respectively, of the distal cap 16, which has the same length L₁ and width W₂ as the proximal body 14. A pair of electrode-receiving bores 54, 56 extend through the planar top and bottom surfaces 51, 53 of the distal cap 16.

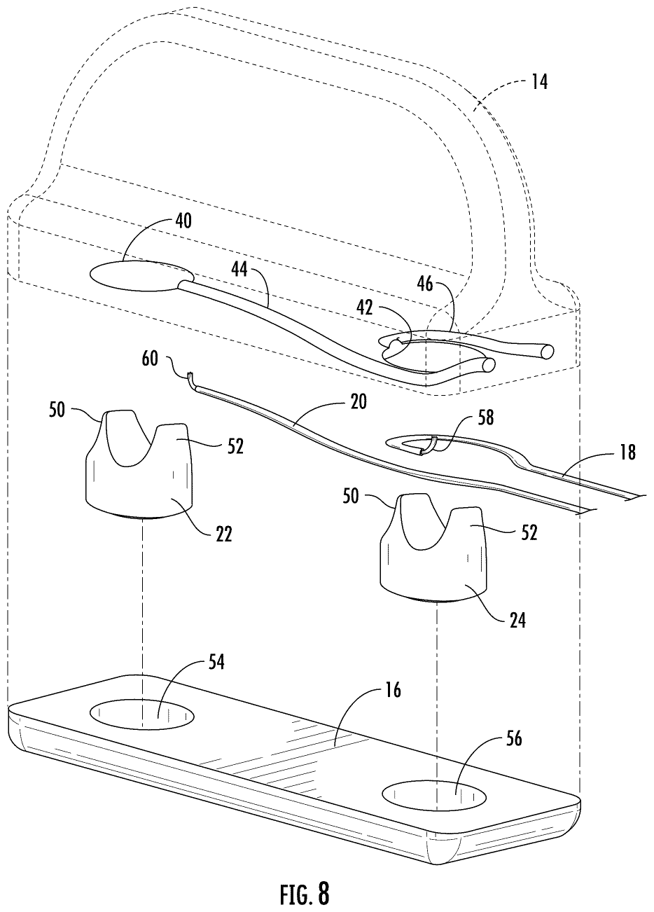

FIG. 8 shows the various elements of the VNS stimulation applicator 10 in exploded relationship to one another. To assemble the applicator 10, the lead wires 18, 20 are pressed into the channels 44,46 on the bottom surface 38 of the base 38 of the proximal body 14. The insulation is stripped from the distal ends of the lead wires 18, 20, exposing free strands 58, 60, which are inserted into the annular cavities 40, 42. The ears 50, 52 of the electrodes 22, 24 are then pressed into the cavities 40, 42, trapping the strands 58, 60 between the ears 50, 52 and the surrounding cavity walls, thus locking the cables 18, 20 in place. Finally, the distal cap 16 is lid, pushed, or pressed over the distal ends of the electrodes 22, 24 and secured to the proximal body 14 by adhesive or solvent bonding, welding, or other attachment methods. A tight seal between the distal cap 16 and proximal body 14 protects the lead wires 18, 30 and electrodes 22, 24 from exposure to dust, liquids, and other potentially harmful substances.

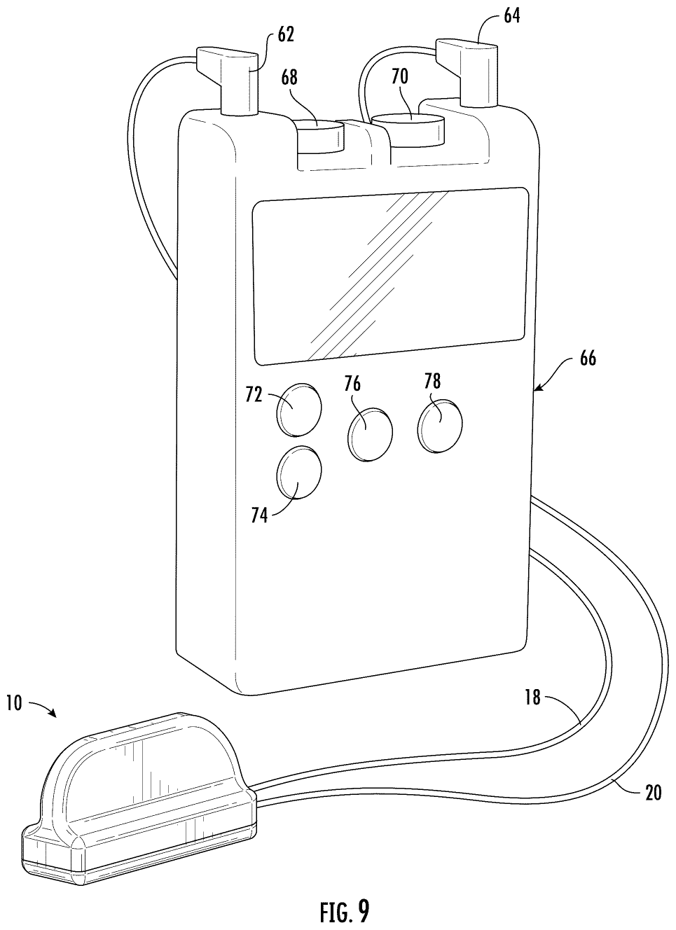

To use the device 10, one simply connects the proximal ends 62, 64 of the lead wires 18, 20 into a programmable TENS unit 66, as shown in FIG. 9, and then holds the device onto the neck at the carotid pulse location for a specified number of minutes. This type of unit 66, which is readily available online and elsewhere without prescription or subscription, may include several dials or control knobs 68, 70, and buttons, 72, 74, 76, 78, which can be used to control the width, frequency, amplitude and other characteristics of the electrical pulses it generates, as well as the duration of treatment. Typically, the pulses are in the form of square waves, although some devices may be programmed to generate other waveforms such as sine waves. These programmable TENS units give users optimum adaptability for adjusting the treatment settings according to the latest research for various disease conditions.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A hand-held vagus nerve stimulation applicator comprising:
a housing including
a proximal body, and
a distal cap having a distal surface configured to be placed on the carotid-pulse area of a user's neck;
a pair of metal electrodes extending through the distal cap and a portion of the proximal body, wherein the size and spacing of the electrodes is optimized for use on the carotid-pulse area of the user's neck;
a pair of lead wires captured between the proximal body and the distal cap and configured to couple the electrodes to a programmable transcutaneous nerve stimulation unit.

2. The vagus nerve stimulation applicator according to claim 1, wherein the distal surface of the distal cap is planar.

3. The vagus nerve stimulation applicator according to claim 2, wherein:
the housing has a predetermined height H₁;
the distal surface has a predetermined length L₁; and
the length L₁ of the distal surface is greater than the height of the height H₁ of the housing.

4. The vagus nerve stimulation applicator according to claim 1, wherein:
the proximal body comprises a distal surface including
a pair of cavities, each cavity configured to receive and retain a proximal portion of one of the metal electrodes, and
a pair of channels, wherein each of the channels communicates with one of the electrodes and is configured to receive and retain one of the lead wires.

5. The vagus nerve stimulation applicator according to claim 1, wherein the distal cap is sealed to the proximal body.

6. The vagus nerve stimulation applicator according to claim 3, wherein:
the proximal body has a distal portion having a predetermined width W₂, and
a proximal portion configured as a finger grip sized and shaped to be easily clasped between an index finger and thumb of a user's hand, the finger grip having a predetermined width W₁ that is less than W₂.

7. The vagus nerve stimulation applicator according to claim 6, wherein W₂ is approximately one half of W₁.

8. The vagus nerve stimulation applicator according to claim 4, wherein:
each of the lead wires has a distal end captured between the proximal portion of one of the electrodes and a wall of the surrounding cavity.

9. The vagus nerve stimulation applicator according to claim 8, wherein:
each of the electrodes includes a solid cylindrical main portion and a pair of diametrically opposed, proximally extending ears; and
the distal end of each lead wire is captured between one of the ears and a wall of the surrounding cavity.

10. A hand-held vagus nerve stimulation applicator comprising:
a housing having
a proximal body including
a distal portion having a predetermined width W₂, and
a proximal portion configured as a finger grip sized and shaped to be easily clasped between an index finger and thumb of a user's hand, the finger group having a predetermined width W₁ that is less than W₂;
a distal cap having a planar distal surface configured to be placed on the carotid-pulse area of a user's neck, the distal surface having the same predetermined width W₂ as the distal portion of the proximal body;
a pair of electrodes extending through the distal cap and a portion of the proximal body, wherein the size and spacing of the electrodes is optimized for use on the carotid-pulse area of the user's neck;
a pair of lead wires captured between the proximal body and the distal cap and configured to couple the electrodes to a programmable transcutaneous nerve stimulation unit.

11. The hand-held vagus nerve stimulation applicator according to claim 10, wherein:
the housing has a predetermined height H₁;
the distal surface has a predetermined length L₁; and
the length L₁ of the distal surface is greater than the height of the height H₁ of the housing.

12. The hand-held vagus nerve stimulation applicator according to claim 11, wherein the finger grip has a predetermined height H₂ that is about two-thirds of H₁.

13. The hand-held vagus nerve stimulation applicator according to claim 12, wherein W₁ is approximately one half of W₂.

14. The hand-held vagus nerve stimulation applicator according to claim 10, wherein the electrode is formed from metal.

15. The hand-held vagus nerve stimulation applicator according to claim 10, wherein:
the proximal body comprises a distal surface including
a pair of cavities, each cavity configured to receive and retain a proximal portion of one of the electrodes, and
a pair of channels, wherein each of the channels communicates with one of the electrodes and is configured to receive and retain one of the lead wires.

16. The vagus nerve stimulation applicator according to claim 15, wherein:
each of the lead wires has a distal end captured between the proximal portion of one of the electrodes and a wall of the surrounding cavity.

17. The vagus nerve stimulation applicator according to claim 16, wherein:
each of the electrodes includes a solid cylindrical main portion and a pair of diametrically opposed, proximally extending ears; and
the distal end of each lead wire is captured between one of the ears and a wall of the surrounding cavity.

18. The vagus nerve stimulation applicator according to claim 10, wherein the distal cap is sealed to the proximal body.
